(19) United States
(12) Patent Application Publication
(10) Pub. No.: US 2004/0213850 A1
(43) Pub. Date: Oct. 28, 2004

(54) Sustained release delivery of a thrombin inhibitor

(76) Inventors: Eleni Dokou, Lansdale, PA (US); Sally Harding, Impington (GB); Shyam B. Karki, Lansdale, PA (US); John Kirsch, Waynesburg, PA (US); Maneesh Nerurkar, Lansdale, PA (US); Kendal G. Pitt, Welwyn Graden City (GB)

Correspondence Address: MERCK AND CO INC, P O BOX 2000, RAHWAY, NJ, 070650907

(21) Appl. No.: 10/829,737

(22) Filed: Apr. 22, 2004

Related U.S. Application Data
(60) Provisional application No. 60/465,713, filed on Apr. 25, 2003.

(51) Int. Cl. A61K 9/24

(52) U.S. Cl. 424/472

(57) ABSTRACT

The invention is an oral pharmaceutical composition comprising

a) a tablet core comprising a therapeutically effective amount of 3-fluoro-2-pyridylmethyl-3-(2,2-difluoro-2-(2-pyridyl)ethylamino)-6-chloropyrazin-2-one-1-acetamide, or a pharmaceutically acceptable salt thereof, a water swellable polymer, and a neutralizing agent, and

b) a water insoluble film coating surrounding the tablet core,

wherein the water insoluble film coated tablet core has a plurality of apertures.

### BACKGROUND OF THE INVENTION

The invention relates to compositions which provide sustained release of a thrombin inhibitor.

Therapeutic compounds are often released quickly in the gastrointestinal tract, leading to sharp increases and subsequent relatively significant decreases in plasma level concentrations. The “Cₘₐₓ/Cₜᵢₘₑ” plasma concentration ratio is a value, at a specified time, calculated by dividing the maximum plasma level concentration by the plasma level concentration at the specific time. The value is useful for representing the speed with which a compound, administered to a patient, dissipates from the biological system. Compounds with high Cₘₐₓ/Cₜᵢₘₑ ratios may not provide the needed sustained, safe and efficacious therapeutic benefit. Such delivery poses potential safety concerns for certain drugs that are not well-tolerated at high levels.

Sustained release formulations have been prepared in a number of ways, generally to protect the active ingredient from exposure to stomach and intestine contents prior to the desired time of release. Typical of such formulations is the one described in U.S. Pat. No. 5,171,580 ('580).

The '580 patent is concerned with a small, three layered sustained release formulation that provides targeted delivery of a cancer drug to the colon, the site where drug release is desired. After the '580 compositions pass from the stomach into the intestine, three layers must dissolve before the drug is absorbed: the outer gastro-resistant coating, which will begin to dissolve once exposed to upper intestine pH of about 5.5; the intermediate gelling layer which swells and builds up a thick gel layer, allowing for delay in the dissolution of the formulation while the formulation is transported down the intestinal path, and; the inner anionic copolymer layer which is soluble at a pH above 7, ensuring drug release in the lower part of the intestinal tract. At column 8, lines 47-55, the '580 patent states that, following administration to a patient, the tablets cover the whole length of the small bowel within 10 hours, without significant changes of the coating and no drug release. Drug is released only in the caecum and colon, when the tablets start to disintegrate. The formulations described in the '580 patent do not release drug in an acidic environment.

U.S. Pat. No. 4,839,177 describes a tablet having a) a deposit core which includes the active ingredient, between 5 and 80% of a crosslinked insoluble swellable polymer such as sodium carboxymethylcellulose or an acrylate, and between 90 and 10% of a gellable polymer such as methylcellulose, carboxymethylcellulose or a glycol, and b) a support platform including a polymeric insoluble material such as a cellulose or acrylate. The polymeric insoluble material also optionally coats the deposit core to modify release properties. The tablet relies on the swelling force of the swellable polymer to control the release of the active ingredient. Swelling force is increased with increasing amounts of swellable polymer and decreases with increasing amounts of gellable polymer. These formulations have the disadvantage of being unable to effectively control release of highly water-soluble active ingredients.

U.S. Pat. No. 4,218,433 describes a tablet having a hollowed portion of specified dimensions which affects elution of water-soluble, slightly water-soluble or water insoluble active ingredient from the tablet. The tablet with the hollowed out portion is coated with an agent that is insoluble in water, water permeable, and soluble in organic solvents, e.g., methacrylic acid ester copolymer. When the tablet is coated, a small space is formed between the film formed by the coating material and the hollow surface portion. The film becomes porous to allow the active ingredient to elute out at a constant rate. If the hollow is smaller that the specified dimensions, the active ingredient won't be eluted from the hollow, and if the hollow is larger than the specified dimensions, the hollow becomes covered with the coating film.

U.S. Pat. No. 4,814,182 discloses the use of rods or slabs of pre-hydrated and swelled polyethylene oxide hydrogel. The polymer is impregnated with a biologically active agent during the hydration procedure. The hydrated polymer is then dried and partially coated with an impermeable, insoluble material. When placed in an aqueous environment, the polymer swells but does not dissolve or disintegrate. The entrapped active ingredient is released form the polymer by diffusion. The mechanism of release is based on the ability of the soluble drug to diffuse through the rehydrated hydrogel and move into the aqueous environment.

An osmotic dosage form which utilizes a semipermeable wall containing at least one “exit means” which passes through the wall, surrounding a core containing an osmotic agent, a neutral and ionizable hydrogel and an active ingredient is taught in U.S. Pat. No. 4,971,790. The coating of this device is permeable to water from the environment of use. Water moves into the core through the semipermeable membrane. Once inside the device, the water solubilizes the osmotic agent, and hydrates the hydrogels. Pressure builds up inside the device. Ultimately, the solubilized hydrogel, containing the beneficial agent, and other core excipients are pumped out of the core, under pressure, through an exit means and into the environment of use.

A frequently encountered problem in the field of sustained release compositions is that many water-miscible drugs have a tendency to be dumped or surged into the body during the first hour or two after an oral dosage form is ingested. This problem is particularly acute when the sustained release compositions are administered with food. Several U.S. Pat. Nos. 4,789,549, 4,816,264 and 4,851,233, have disclosed devices that have an improved sustained release activity. However, none are entirely satisfactory since they have a tendency to rapidly release water-miscible drugs when administered with food. Additionally, the devices disclosed are not insensitive to the pH of the environment of use.

U.S. Pat. No. 4,327,725 ('725) discloses an osmotic device which has a wall formed of a semipermeable material permeable to the passage of an exterior aqueous fluid and substantially impermeable to passage of an active agent, and a passageway in the wall communicating with the active agent an the exterior of the device.

U.S. Pat. No. 5,057,321 ('321) discloses a dosage form for delivering a drug to an animal, comprising a wall, permeable at least in part to the passage of fluid, which surrounds a maltodextrin-containing compartment, and an exit passageway in the wall.

U.S. Pat. No. 5,366,738 ('738) discloses a device which consists essentially of a homogeneous compressed core prepared from an admixture comprising a therapeutically effective amount of a pharmaceutically active ingredient, and a polymer which forms microscopic gelatinous beads upon hydration. The core is coated with a water insoluble, water impermeable polymeric coating, which surrounds and adheres to the core, the coating having a plurality of apertures, exposing between about 5 and about 75% of the core surface.

WO 00/75134 describes pyrazinone thrombin inhibitors, e.g. 3-fluoro-2-pyridylmethyl-3-(2,2-difluoro-2-(2-pyridyl)ethylamino)-6-chloropyrazin-2-one-1-acetamide, and pharmaceutically acceptable salts thereof, which are useful for treating and preventing diseases and conditions mediated by thrombin inhibitors such as thrombus formation, deep vein thrombosis, and related conditions. The inhibitors are described as suitable for administration in oral forms such as tablets, capsules (including sustained release forms), pills, powders, granules, elixers, tinctures, suspensions, syrups, and emulsions, and other administration forms, including intravenous (bolus or infusion), intraperitoneal, subcutaneous, topical (e.g., transdermal, intraocular), intranasal and intramuscular forms.

The compositions of the present invention solve the difficult problem of providing therapeutically safe and effective sustained plasma level concentration of a thrombin inhibitor by reducing the time-dependent peak-to-trough ratio. The compositions of the invention act to control release of the thrombin inhibitor by regulating both diffusion and extrusion of the drug from the tablet and into the system.

### SUMMARY OF THE INVENTION

The invention is an oral pharmaceutical composition comprising

a) a tablet core comprising a therapeutically effective amount of 3-fluoro-2-pyridylmethyl-3-(2,2-difluoro-2-(2-pyridyl)ethylamino)-6-chloropyrazin-2-one-1-acetamide, or a pharmaceutically acceptable salt thereof, a water swellable polymer, and a neutralizing agent, and

b) a water insoluble film coating surrounding the tablet core,

wherein the water insoluble film coated tablet core has a plurality of apertures.

3-Fluoro-2-pyridylmethyl-3-(2,2-difluoro-2-(2-pyridyl)ethylamino)-6-chloropyrazin-2-one-1-acetamide, and pharmaceutically acceptable salts thereof, are hereinafter also referred to as “active ingredient.”

In one class of compositions, the tablet core comprises an amount of 3-fluoro-2-pyridylmethyl-3-(2,2-difluoro-2-(2-pyridyl)ethylamino)-6-chloropyrazin-2-one-1-acetamide of between about 10% and about 60% by weight of the total core mass, an amount of neutralizing agent between about 10% and about 50% by weight of the total core mass, an amount of water swellable polymer between about 5% and about 30% by weight of the total core mass, in addition to other tablet excipients. The tablet core is coated with a water insoluble film coating comprising between about 60% and about 95% film forming polymeric material and between about 5% and about 30% plasticizer. The coating thickness is between about 10 and about 1000 microns. Between about 6 and about 200 apertures, each having a diameter of between about 0.1 mm and about 0.9 mm, are drilled through the water insoluble film coating, using, for example, a laser driller. For a two-sided tablet for example, between about 3 and 100 apertures per tablet side are drilled.

The invention also comprises a method for orally administering, to a patient, a therapeutically effective dose of the active ingredient in a pharmaceutical composition core-comprising the active ingredient, a water swellable polymer, and a neutralizing agent, wherein the core is coated with a water insoluble film coating material surrounding the tablet, and wherein said composition has a plurality of apertures which provide a means for the active ingredient to leave the core unimpeded by the film.

Compositions of the invention are useful in the manufacture of a medicament for sustained release of the active ingredient.

### DETAILED DESCRIPTION OF THE INVENTION

By “pharmaceutically active agent,” “active ingredient,” “medicament,” or “beneficial agent” is meant 3-fluoro-2-pyridylmethyl-3-(2,2-difluoro-2-(2-pyridyl)ethylamino)-6-chloropyrazin-2-one-1-acetamide, and pharmaceutically acceptable salts thereof, and derivatives that produce similar localized or systemic effect or effects in animals. Derivatives of the active ingredient, such as esters, ethers and amides without regard to their ionization and solubility characteristics can be used alone or mixed with other compounds. Also, prodrugs of the active agent can be used in a form that, upon release from the tablet, is converted by enzymes, hydrolyzed by body pH or converted by other metabolic processes, to the original form, or to a biologically active form. That is, prodrugs are specifically included within the definition of pharmaceutically active ingredients. Some of the active ingredients included within the compositions of the present invention are chiral; included within the scope of the present invention compositions are those having racemic mixtures and separated enantiomers of the active ingredient. Furthermore, hydrates as well as anhydrous compositions and polymorphs of the active ingredient may be included in compositions of the present invention.

The term “pharmaceutically acceptable salts” means non-toxic salts of the active ingredients which are generally prepared by reacting the free base with a suitable organic or inorganic acid. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, ma ndelate, mesylate, methylbromide, methylnitrate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, valerate.

The terms “water swellable polymer”, “water swelling polymer”, and “gel forming polymer” all refer to a polymer used in the tablet core that is capable of swelling upon hydration. The polymer is capable of producing discrete gel particles which support a suspension, including the beneficial agent, as it forms. The term “swellable” implies that the polymer is in a non-hydrated state, while the term “swelling” implies that the polymer is in a hydrated state. The gel forming polymer used also must exude from the core surface in such a way that the beneficial agent is carried into the environment of use. Upon hydration, the gel particles must be predisposed to leave the surface taking the drug with it. This assures a constant surface area exposed to the solvent of the environment of use and maintains the appropriate rate of release. The gel particles are composed of discrete particles of hydrated polymer. Both size and hydration rate of these microscopic gel particles are characteristics of the hydrated polymer. Illustrative of swellable polymer which forms gel particles are “AQUAKEEP J-550”, “AQUAKEEP J-400”, which are trade names for sodium acrylate polymer produced by Seitetsu Kagaku Co., Ltd., Hyogo, Japan. The “AQUAKEEP” polymers are generically described in U.S. Pat. No. 4,340,706. Also illustrative of this type of polymer are the carboxypolymethylenes prepared from acrylic acid cross-linked with allyl ethers of sucrose or pentaerythritol and sold under the trade names “CARBOPOL 934P” and “CARBOPOL 974P” which are trade names for two carbamer type polymers produced by B.F. Goodrich Chemical Company, Cleveland, Ohio. Carbamer polymers are generically described in U.S. Pat. No. 2,909,462 and in the National Formulary XVII at p. 1911, CAS Registry Number 9003-01-4. All of the forgoing references are hereby incorporated by reference. In the dry state, “CARBOPOL 974P” and “CARBOPOL 934P” particles range in size from 2 to 7 microns. When these dry particles are hydrated, microscopic gel particles in the range of 20 microns are produced. When “AQUAKEEP J-550” or “AQUAKEEP J-400” dry particles are hydrated, microscopic gel particles diameter can range in size from 100 to 1000 microns.

The “neutralizing agent” modulates swellable polymer hydration and provides for release of the active ingredient from the tablet core into the gastrointestinal tract by diffusion directly from the core and by extrusion of swelling polymer. The neutralizing agent is solubilized by the aqueous media of the environment and establishes an environment such that the environment pH, ionic strength or hydrophilic character is appropriate for the desired polymer gel particle hydration rate. For example, the neutralizing agents can enhance or retard the neutralization of acidic functional groups on the polymer, thereby affecting the hydration rate. Among the groups of compounds that can exert this effect are bases and the salts of bases such as sodium carbonate, sodium bicarbonate, betaine hydrochloride, sodium citrate, arginine, meglamine, sodium acetate, sodium phosphates (e.g., sodium phosphate dibasic anhydrous), potassium phosphates, calcium phosphate, ammonium phosphate, magnesium oxide, magnesium hydroxide, sodium tartrate and tromethamine. Other compounds that can be used as polymer hydration modifiers include sugars such as lactose, sucrose, mannitol, sorbitol, pentaerythritol, glucose and dextrose. Polymers such as microcrystalline cellulose and polyethylene glycol, as well as surfactants and other organic and inorganic salts can also be used to modulate polymer hydration. Most preferably, sodium phosphate dibasic anhydrous is used.

By “drug delivery device” is meant, a dosage form that provides a convenient means of delivering a pharmaceutically active ingredient or drug to a subject in need thereof. The subject can be a human or any other animal in need of such pharmaceutically active ingredient. The device is designed to be useful for the delivery of a pharmaceutically active ingredient by any pharmaceutically accepted means such as by swallowing, retaining it within the mouth until the beneficial agent has been dispensed, placing it within the bucal cavity, or the like.

By “controlled” is meant that the rate of release of the pharmaceutically active ingredient from the device to the environment of use is not immediate, but rather, follows a predetermined pattern. Thus, relatively constant or predictably varying amounts of the beneficial agent can be dispensed over a specified period of time.

The term “animal” includes mammals, humans and primates such as domestic, household, sport or farm animals such as sheep, goats, cattle, horses and pigs, laboratory animals such as mice, rats and guinea pigs, fishes, avians, reptiles and zoo animals.

By “compressed core” is meant an admixture of ingredients comprising an active ingredient, a water swellable polymer which produces gel particles when hydrated, neutralizing agent, and other ingredients that may affect the rate of production of the dispersion, the stability of the components of the dosage form, or the mixing or compression characteristics of the admixture, blended in such a way to produce a uniform material that is compressed, within a die, to produce a desired form, normally in the shape of a tablet, capsule or bolus.

The compressed core contains a “therapeutically effective amount” of active ingredient. By “therapeutically effective amount” is meant that the quantity of pharmaceutically active ingredient which has been demonstrated to be sufficient to induce the desired effect during studies utilizing the compound.

The formed gel particles are a semisolid system consisting of hydrated polymer interpenetrated by the aqueous solvent of the environment of use.

Tablet Core

In one embodiment of the tablet core, the amount of active ingredient is between about 10% and about 60% by weight of the total core mass (e.g., between 5 mg and 500 mg or more, e.g. 5 mg, 50 mg, 100 mg, 150 mg, or 225 mg), the amount of neutralizing agent is between about 10% and about 50% by weight of the total core mass, and the amount of water swellable polymer is between about 5% and about 30% by weight of the total core mass.

The tablet core is typically in the form of a solid conventional tablet. Generally, the core is compressed into its final shape using a standard tablet compressing machine. The core may contain compressing aids and diluents such as lactose that assist in the production of compressed tablets. The core can be comprised of a mixture of agents combined to give the desired manufacturing and delivery characteristics. The number of agents combined to make the core is substantially without an upper limit, and with a lower limit of two agents: the gel forming polymer, and the active ingredient. Preferably, a third agent, which is a neutralizing agent, is used.

The active ingredient may be in a layer of the core as a dispersion, particle, granule, or powder. Also, the pharmaceutically active ingredient may be mixed with a binder, dispersant, emulsifier or wetting agent and dyes. Other excipients such as lactose (e.g. Lactose NF hydrous spray dry), magnesium stearate, microcrystalline cellulose (e.g. Avicel PH 101), starch, stearic acid, calcium phosphate, glycerol monostearate, sucrose; polyvinylpyrrolidone, gelatin, methylcellulose, sodium carboxymethylcellulose, sorbitol, mannitol, polyethylene glycol and other ingredients commonly utilized as stabilizing agents or to aid in the production of tablets may also be present in the layers of the core.

In instances where the pharmaceutically active ingredient, the gel forming polymer and neutralizing agent exhibit the desired release rate, stability, and manufacturing characteristics, there is no critical upper or lower limit as to the amount of pharmaceutically active ingredient that can be incorporated into a layer of the core. The ratio of drug to excipient is dictated by the desired time span and profile of release, and the pharmacological activity of the drug.

Once the core is prepared, it can be coated and drilled in the manner described.

Tablet Coating

The “water-insoluble film coating” surrounding the tablet core is formed by a film forming polymeric material. The coating, applied to the tablet core, is a material that is insoluble in the environment of use, can form films, and does not adversely affect the pharmaceutically active ingredient, animal body, or host. The coating is insoluble in water and impermeable to the selected product, drugs, neutralizing agent, or to other compounds in the device. This coating is insoluble in body fluids and non-erodible or it can be bioerodible after a predetermined period with bioerosion following the end of the active drug release period. In each instance, it is insoluble to solvent and solute(s) found in the environment of use and is suitable for construction of the device. The coating can be formed by dipping the cores into a suitable solution of the film forming polymeric material or by spray coating the cores with the solution. The film-forming polymers used in the coating which are herein described are known to the art or can be prepared according to the procedures in the Encyclopedia of Polymer Science and Technology, Vol. 3, published by Interscience Publishers, Inc., New York, in Handbook of Common Polymers by Scott, J. R. and Roff, W. J., 1971, published by CRC Press, Cleveland, Ohio. Among the film forming polymeric materials that can provide this type of protection are cellulose acetate, cellulose acetate butyrate (e.g. cellulose acetate butyrate 381-20), ethylcellulose, polyvinylacetate, polyvinyl chloride and polymers of acrylic and methacrylic acid esters. In addition, other materials, such as plasticizers, may be included with the coating to enhance its stability, color, elasticity, flexibility, ease of application or opacity. Types of plasticizers that may be used include, but are not limited to, dibutylsebacate, diethyl phthalate, triethyl citrate (e.g. triethyl citrate PG/NF) and polyethylene glycol. Preferably, the polymer comprises polyvinyl chloride, cellulose acetate, cellulose acetate butyrate or ethylcellulose, or combinations thereof. Preferably, the plasticizer comprises diethylphthalate, dibutylsebacate or triethylcitrate. The solutions for spraying or dipping are formed with solvents such as acetone or alcohol or a combination of acetone and alcohol. The alcohol can be, for example, ethyl dehydrated 200 proof alcohol.

The coating is applied to a thickness of from about 10 to about 1000 microns, e.g. 100, 250, 500 and 750 microns, but preferably from about 100 to about 500 microns. The coating is formed over the entire surface of the core. Apertures are cut in the coating to expose the core, using either a mechanical or laser drill, a coring device or any other pharmaceutically accepted means. In one embodiment, a mechanical drill is used to produce the apertures. In another embodiment, a laser is used to make the apertures.

Apertures

The expression “aperture” as used herein, refers to ports or holes through the coating which expose the surface of the core to the environment. The apertures (e.g. holes drilled by a laser driller) provide controlled release of the active ingredient from the tablet core, unimpeded by the film. The apertures allow solution to make contact only with exposed portions of the core when in use. The number, size and configuration of the apertures is chosen to provide the release rate required to suit a pharmacologically recognized requirement since the hydration of the polymer will occur only where the apertures allow such core-solvent contact. The size and number of apertures is chosen to effect the desired release rate. In determining the aperture size and number, the hydration rate of the gel forming polymer, the type and concentration of the neutralizing agent used in the core and the ability of the beneficial agent to form ions must be considered. The apertures are generally positioned in a regular pattern on both faces of the device although they can be positioned anywhere on the core including the edges or as previously described, on one face. The apertures are generally circular but may be of any design that results in the proper release rate. When the aperture is circular, its diameter ranges from about 0.1 mm to about 0.9 mm with diameters of about 0.5 mm being typical. The total number of apertures may be between 6 and 200, typically between 10 and 160, e.g. 20, 30, 36, 40, 60, 62, 80, 100, and 160. Apertures may be drilled on both sides of a two-sided (two-faced) tablet, having, for example, 10, 15, 18, 20, 30, 31, 40 or 80 apertures per face.

The apertures may be made by drilling the appropriate size hole through the coating using a mechanical or laser based process. In the preferred embodiment, a digital laser marking system is used to drill the holes required. This system allows for an array of apertures to be drilled on both faces of a dosage form simultaneously and at rates suitable for production of dosage forms.

The steps involved in the laser drilling process are as follows: a digital laser marking system is focused at a laser stage; the dosage form is moved onto the laser stage of the digital laser marking system; the digital laser marking system is pulsed to energize those laser tubes needed to drill the desired apertures along a linear array on the dosage form; the dosage form is moved forward on the laser stage and the digital laser marking system is again pulsed as needed to produce an additional linear array of apertures. The dosage form is then removed from the laser stage.

One embodiment of the insoluble film includes a mixture of eight parts by weight of cellulose acetate butyrate, two parts by weight of cellulose acetate and one part by weight of diethylphthalate. This mixture is dissolved in a solution of methylene chloride and methanol (3:1 v/v) and sprayed onto the cores to a thickness of about 250 microns. Another preferred coating consists of nine parts by weight of cellulose acetate butyrate and one part by weight of triethyl citrate dissolved in a mixture of acetone and methanol (3:1 v/v). This mixture is sprayed on the core or the cores are dipped into the mixture so that a coating of about 100 microns is applied.

In a preferred embodiment of the impermeable wall, a mixture of nine parts by weight of cellulose acetate butyrate and one part by weight of triethylcitrate is dissolved at 5% w/v in a solution of acetone and ethanol (3:1 v/v) or methylene chloride and methanol (3:1 v/v) and sprayed onto the cores to a thickness of about 100 microns.

Once the composition is within the environment of use, the polymer of the compressed core which is exposed to the ambient aqueous solution at the coating apertures begins to hydrate and produce gel particles. During the production of the swelling polymer, soluble and insoluble core components located near the polymer particles become dispersed and mixed in such a manner that a gelatinous dispersion is produced. The dispersion moves a portion of the active ingredient from the core into the aqueous solvent, bringing the beneficial agent into the environment of use. Another portion of active ingredient move into the environment free of interaction with the swelling polymer.

In operation, aqueous solution from the environment of use (e.g., gastrointestinal system) contacts the surface of the core that is exposed within the apertures. The available water begins to hydrate the polymer at the surface of the core. The neutralizing agent, at the exposed core surface, is solubilized and establishes the environment required for control of polymer hydration. As the polymer particles are hydrated, the swelling polymer moves from the surface. At the same time, the swelling polymer moves a portion of the active ingredient from the surrounding surface by extrusion into the environment. The swelling polymer moves from the core surface into the environment of use in a dispersion. Another portion of the active ingredient moves from the core into the environment by diffusion. As a result, controlling the surface area of the core which is exposed to the environment of use, the portion of active ingredient moved into the environment by the swelling polymer, and the portion of active ingredient diffused directly into the environment, effectively controls the delivery rate of medicament to the environment. The neutralizing agent regulates gel formation at a rate which allows for a portion of the highly water-soluble active ingredient to become integrated within the gel matrix formed by the polymer, and allows for another portion to freely move from the core directly into the aqueous environment. Delivery of the active ingredient occurs from the surface of the core and from within the core so that the delivery rate is dependent on diffusion of the active ingredient from inside the core to the environment of use as well as extrusion of the swelling polymer, containing the active ingredient, from the core.

The following examples illustrate the preparation of the drug delivery device of this invention and the controlled release of active ingredient into an environment of use, and are not to be considered as limiting the invention set forth in the claims appended hereto.

The dosage regimen utilizing the compositions of the present invention is selected in accordance with a variety of factors including type, species, age, weight, sex and medical condition of the patient; the severity of the condition to be treated; the route of administration; the renal and hepatic function of the patient; and the particular active ingredient or salt thereof employed. An ordinarily skilled physician or veterinarian can readily determine and prescribe the effective amount of the drug required to prevent, counter, or arrest the progress of the condition.

In the following example, 3-fluoro-2-pyridylmethyl-3-(2,2-difluoro-2-(2-pyridyl)ethylamino)-6-chloropyrazin-2-one-1-acetamide is the active ingredient. The drug is highly effective in the inhibition of thrombin in humans.

### EXAMPLE 1

### Compositions containing 3-fluoro-2-pyridylmethyl-3-(2,2-difluoro-2-(2-pyridyl)ethylamino)-6-chloropyrazin-2-one-1-acetamide

Tablets containing 50, 100, 150 and 225 mg respectively, of the compound 3-fluoro-2-pyridylmethyl-3-(2,2-difluoro-2-(2-pyridyl)ethylamino)-6-chloropyrazin-2-one-1-acetamide were prepared using the following ingredients and amounts and formulation procedures.

| | % (weight) |
|---|---|
| Tablet Core Ingredients | |
| 3-fluoro-2-pyridylmethyl-3-(2,2- | 37.5% |
| difluoro-2-(2- | |
| pyridyl)ethylamino)-6- | |
| chloropyrazin-2-one-1- | |
| acetamide | |
| Sodium phosphate dibasic | 30 |
| anhydrous | |
| Lactose NF hydrous spray dry | 16 |
| Carbopol 974 P | 15 |
| Magnesium stearate | 1.5 |
| | 100% |
| Film Coating Ingredients | |
| Cellulose acetate butyrate 381-20 | 4.55 |
| Triethyl citrate, PG/NF | 0.45 |
| Ethanol dehydrated 200 proof* | 23.75 |
| Acetone NF* | 71.25 |
| | 100% |

*these excipients are removed during processing

3-fluoro-2-pyridylmethyl-3-(2,2-difluoro-2-(2-pyridyl)ethylamino)-6-chloropyrazin-2-one-1-acetamide (active ingredient), Carbopol 974P (swellable polymer), sodium phosphate dibasic anhydrous (neutralizing agent) and lactose NF hydrous spray dry (tablet excipient) are placed in a V blender and mixed for 5 minutes, compacted into ribbons and milled. The milled material is lubricated with magnesium stearate (tablet excipient) and compressed into tablets.

The cores are coated with the impermeable wall a mixture of cellulose acetate butyrate 381-20 (film forming polymeric material) and triethyl citrate PG/NF (plasticizer). This mixture, at 5% w/v, is dissolved in a solution of acetone NF and ethanol dehydrated 200 proof (3:1 v/v) and sprayed onto the cores to obtain 7% of the finished tablet weight as coating, providing a thickness of about 100 microns, using a Freund Model HCT-Mini Hi-Coater (pan).

The total weight of finished tablets as a function of amount of active ingredient is as follows:

| Active ingredient | Total tablet weight with coating |
|---|---|
| 50 mg | 142.6 mg |
| 100 mg | 285.4 mg |
| 150 mg | 428 mg |
| 225 mg | 642 mg |

A laser driller is used to drill 0.5 mm holes through the coating to the surface on both tablet faces. Tablets containing 50 mg of active ingredient were prepared with 10 holes/face, 18 holes/face or 31 holes/face. Dissolution testing showed that 80% release was achieved in 8 hours with 31 holes per tablet face, 12 hours with 18 holes per tablet face, and 16 hours with 10 holes per tablet face.

Tablets were orally administered to patients. Table 1 shows that active ingredient was released in a sustained manner compared to immediate release (IR) tablets. Table 2 shows that active ingredient plasma level concentration over time was maintained at a moderate level, with decreased peak concentration, compared to immediate release tablets.

Table 3 shows substantially controlled Cₘₐₓ/Cₜᵢₘₑ ratios, demonstrating safe and effective plasma level concentrations over extended periods of time. The ratio is calculated by dividing the maximum plasma level concentration by the plasma level concentration at a particular time. Cₘₐₓ/Cₜᵢₘₑ for the 50 mg tablet with 10 holes on each face rises only slightly over 16 hours, whereas Cₘₐₓ/Cₜᵢₘₑ for the immediate release tablet rises sharply after 4 hours. These data demonstrate that safe and effective dosing of the active ingredient can be achieved using the formulation of the present invention.

TABLE 1
% Active ingredient release from 50 mg tablet (0.5 mm holes)

| Hours | 10 holes/face | 18 holes/face | 31 holes/face | IR tablet |
|---|---|---|---|---|
| 2 | 12 | 18 | 29 | 100 |
| 4 | 28 | 37 | 58 | — |
| 6 | 42 | 54 | 72 | — |
| 8 | 55 | 66 | 80 | — |
| 10 | 64 | 73 | 83 | — |
| 12 | 70 | 79 | 85 | — |
| 14 | 74 | 82 | 86 | — |
| 16 | 78 | 82 | 87 | — |
| 18 | 80 | 83 | 88 | — |

“IR” means immediate release.

TABLE 2
Plasma level concentration (nM) - 50 mg tablet

| Hours | 10 holes/tablet face | IR tablet |
|---|---|---|
| 2 | 110 | 620 |
| 4 | 140 | 520 |
| 6 | 120 | 200 |
| 8 | 100 | 120 |
| 10 | 70 | 80 |
| 12 | 70 | 45 |
| 14 | 65 | 40 |
| 16 | 60 | 40 |

“IR” means immediate release.

TABLE 3
Cₘₐₓ/Cₜᵢₘₑ - 50 mg tablet

| Hours | 10 holes/tablet face | IR tablet |
|---|---|---|
| 2 | 1.3 | 1.0 |
| 4 | 1.0 | 1.2 |
| 6 | 1.2 | 3.1 |
| 8 | 1.4 | 5.2 |
| 10 | 2.0 | 7.8 |
| 12 | 2.0 | 14 |
| 14 | 2.2 | 16 |
| 16 | 2.3 | 16 |

“IR” means immediate release.

Sustained release, decreased peak concentration, and safe and effective plasma level concentration over an extended period of time, was similarly achieved with tablets containing 100, 150 and 225 mg active ingredient.

What is claimed is:

1. An oral pharmaceutical composition comprising
a) a tablet core comprising a therapeutically effective amount of 3-fluoro-2-pyridylmethyl-3-(2,2-difluoro-2-(2-pyridyl)ethylamino)-6-chloropyrazin-2-one-1-acetamide, or a pharmaceutically acceptable salt thereof, a water swellable polymer, and a neutralizing agent, and
b) a water insoluble film coating surrounding the tablet core,
wherein the water insoluble film coated tablet core has a plurality of apertures.

2. The composition of claim 1, wherein the tablet core comprises an amount of 3-fluoro-2-pyridylmethyl-3-(2,2-difluoro-2-(2-pyridyl)ethylamino)-6-chloropyrazin-2-one-1-acetamide between about 10% and about 60% by weight of the total core mass, an amount of neutralizing agent between about 10% and about 50% by weight of the total core mass, and an amount of water swellable polymer between about 5% and about 30% by weight of the total core mass

3. The composition of claim 1, wherein the water insoluble film coating comprises between about 60% and about 95%. film forming polymeric material and between about 5% and about 30% plasticizer.

4. The composition of claim 1, wherein the film coating has a thickness of between about 10 and 1000 microns.

5. The composition of claim 4, wherein the film coating has a thickness of from about 100 to about 500 microns.

6. The composition of claim 1, wherein the plurality of apertures is between about 6 and about 200.

7. The composition of claim 6, wherein the apertures have a diameter of between about 0.1 and about 0.9 mm.

8. The composition of claim 7, wherein the apertures have a diameter 5 of about 0.5 mm.
